(19) United States
(12) Patent Application Publication
(10) Pub. No.: US 2008/0274161 A1
(43) Pub. Date: Nov. 6, 2008

(54) HYDROGELS AND HYDROGEL PARTICLES

(76) Inventors: Orhun K. Muratoglu, Cambridge, MA (US); Gavin J. C. Braithwaite, Cambridge, MA (US); Jeeyoung Choi, Cambridge, MA (US); Stephen H. Spiegelberg, Winchester, MA (US)

Correspondence Address: PROSKAUER ROSE LLP, 1001 PENNSYLVANIA AVE, N.W.,, SUITE 400 SOUTH, WASHINGTON, DC, 20004, US

(21) Appl. No.: 11/419,142

(22) Filed: May 18, 2006

Related U.S. Application Data
(60) Provisional application No. 60/682,008, filed on May 18, 2005.

(51) Int. Cl. A61K 9/14; A61P 43/00

(52) U.S. Cl. 424/425

(57) ABSTRACT

The invention provides fabricated hydrogels, hydrogel particles, hydrogel containing compositions, and methods of making the same. The invention also provides methods of implanting, injecting, or administering the hydrogels, hydrogel particles, or the hydrogel containing compositions to treat a subject in need. Methods of crosslinking pre-solidified or pre-gelled hydrogel particles and making crosslinked hydrogels, crosslinked hydrogel particles, and crosslinked hydrogel containing compositions also are disclosed herein.

This application claims priority to Provisional Application No. 60/682,008 filed on May 18, 2005, which is hereby incorporated by reference.

### FIELD OF THE INVENTION

The invention relates to fabrication of hydrogels, hydrogel particles, hydrogel containing compositions, and methods of administering fabricated hydrogels, hydrogel particles, and hydrogel containing compositions. The invention also relates to methods of making the hydrogels, hydrogel particles, the hydrogel containing compositions, and methods of using the same in treating a subject in need.

### BACKGROUND OF THE INVENTION

Hydrogels are three-dimensional, water-swollen structures composed of mainly hydrophilic homopolymers or copolymers, for example, polyvinyl alcohol (PVA), polyacrylamide (PAAM), poly-N-isopropylacrylamide (PNIAAm), polyvinyl pyrrolidone (PVP). PVA-based hydrogels have been disclosed for use in a variety of biomedical applications, (see Hassan & Peppas, Advances in Polymer Science, vol. 153, Springer-Verlag Berlin Heidelberg, 2000, pp. 37-65; Lowman et al. Ed., John Wiley and Sons, 1999. pp. 397-418).

Hydrogels have been used in a variety of biomedical applications, for example, intervertebral disc replacement or disc augmentation, wound care, cartilage replacement, joint replacement, surgical barriers, gastrointestinal devices, drug, delivery, cosmetic and reconstructive surgery, and breast enlargement.

Hydrogel formulations are also known for their use for injection into body cavities in a liquid form to undergo gelation inside the cavity (see Ruberti and Braithwaite: US Publication Nos. 20040092653 and 20040171740).

Lawman et al. (US Publication No. 2004/0220296) describe a gel formulation, which is also injectable in a liquid form. The liquid formulation undergoes a phase transformation to form a solid hydrogel implant in situ at physiological body temperature.

Another gel formulation has been described by Stedronsky et al. (U.S. Pat. No. 6,423,333). Stedronsky et al. utilized a protein based gel and injected as a fluid into a bodily cavity where it formed a solidified gel.

Sawhney (U.S. Pat. No. 6,818,018) discusses injectable hydrogel formulations that, upon injection into a body cavity, undergo physical associations through chelating agents or thermo-reversible transitions, and then chemically crosslink through the incorporation of crosslinking agents.

Bao and Higham (U.S. Pat. No. 5,192,326) describe a prosthetic nucleus comprised of hydrogel beads contained within a membrane that is semi-permeable to aqueous fluids. The beads contain only the polymer and water, the latter of which comprises at least 30% of the bead content.

In the above described type of systems, the goal is to completely fill a cavity with a liquid gel and allow the gel to solidify in the cavity through a gelation process to achieve a non-flowing gel system occupying the space inside the cavity.

None of the publications described above disclose a hydrogel formulation containing pre-solidified hydrogel particles in a precursor hydrogel solution. Implantation or injection of a hydrogel-particle formulation minimize the amount of fluid gel to be administered into a body cavity and would therefore require only a small proportion of the implantation or precursor hydrogel solution to undergo the gelling/gelationtion process inside the cavity. Other advantages of an implantable or injectable hydrogel-particle system and formulations are discussed below.

In addition, the hydrogel-particles can be used to fabricate hydrogel implants with a variety of desired properties. The hydrogel particles of one kind can be placed in a hydrogel matrix to form a fabricated article, which can be an implant or can be machined to an implant shape. Different formulations of hydrogel particles can be blended and embedded in any hydrogel matrix of a different chemical composition to tailor the properties of the fabricated article or implant. Then, the implant will be surgically administered to a patient in need, for example, to fill a cartilage defect, to fill a nuclear space in an intervertebral disc, to augment a tissue, or to replace a tissue.

Hydrogel-particle systems, formulations, injectable hydrogel-particle systems,

fabricated hydrogel implants containing hydrogel particles, methods of administration and their use in treating a subject in need are disclosed for the first time by the present invention.

### SUMMARY OF THE INVENTION

The present invention relates generally to fabricated hydrogels, hydrogel particles, compositions containing particles, and methods of making and using the same.

The invention provides fabricated hydrogels, hydrogel particles, mechanically deformed hydrogel particles, compressed hydrogel particles, methods of making the hydrogel particles and fabrication of the particulate hydrogel systems. The invention also provides pre-gelled or pre-solidified hydrogel particles that are small enough in size to pass through an injection needle. Size of the needle can vary, for example, a needle size of about 33, about 28, about 25, about 22, about 20, or about 18 gauge or lower, or any size thereabout or therebetween, is preferred. The inner diameter of the needle also can vary, for example, an inner diameter of about 0.025 mm or more, about 0.089 mm or about 0.10 mm or more, or any diameter thereabout or therebetween.

In one aspect, the invention provides fabricated hydrogel particles comprising at least one type of gellant, wherein the gellant is embedded within the hydrogel particles, and wherein the gellant diffuses out of the hydrogel particles, thereby gelling the surrounding hydrogel matrix.

In another aspect, the invention provides fabricated hydrogel particles comprising mechanically deformed hydrogel particles, wherein the hydrogel particles comprising at least one type of gellant, wherein the gellant is embedded within the hydrogel particles, and wherein the gellant diffuses out of the hydrogel particles, thereby gelling the surrounding hydrogel matrix.

In another aspect, the invention provides fabricated hydrogel particles comprising at least one type of gellant, wherein the gellant is embedded within the hydrogel particles, wherein the hydrogel particles are injectable in size, and wherein the gellant diffuses out of the hydrogel particle when the particle is injected to a body cavity, thereby gelling the surrounding hydrogel matrix.

In another aspect, the invention provides fabricated hydrogel compositions comprising: a) fabricated hydrogel particles; b) at least one type of gellant, wherein the to gellant is embedded within the hydrogel particles; and c) a precursor hydrogel solution, wherein the hydrogel particles are suspended in the hydrogel solution, and wherein the gellant diffuses out of the hydrogel particles, thereby gelling the surrounding hydrogel matrix.

In another aspect, the invention provides fabricated hydrogel compositions comprising: a) fabricated hydrogel particles, wherein the hydrogel particles are injectable in size; b) at least one type of gellant, wherein the gellant is embedded within the hydrogel particles; and c) a hydrogel solution, wherein the hydrogel particles are suspended in the precursor hydrogel solution, and wherein the gellant diffuses out of the hydrogel particles when the composition is injected to a body cavity, thereby gelling the surrounding hydrogel matrix.

In another aspect, the invention provides fabricated hydrogel particles comprising: a) polyvinyl alcohol (PVA) particles; b) at least one type of gellant wherein the gellant is embedded within the hydrogel particles, and wherein the gellant diffuses out of the hydrogel particle, thereby gelling the surrounding hydrogel matrix.

In another aspect, the invention provides fabricated hydrogel particles comprising: a) polyvinyl alcohol (PVA) hydrogel particles, wherein the hydrogel particles are injectable in size; b) at least one type of gellant, wherein the gellant is embedded within the hydrogel particle, and wherein the gellant diffuses out of the hydrogel particle when the particle is injected to a body cavity, thereby gelling the surrounding hydrogel matrix.

In another aspect, the invention provides fabricated hydrogel compositions comprising; a) fabricated hydrogel particles, wherein the hydrogel particles comprise PVA; b) at least one type of gellant, wherein the gellant is embedded within the hydrogel particles; and c) a precursor hydrogel solution, wherein the hydrogel particles are suspended in the hydrogel solution, and wherein the gellant diffuses out of the hydrogel particles, thereby gelling the surrounding hydrogel matrix.

In another aspect, the invention provides fabricated hydrogel compositions to comprising; a) fabricated hydrogel particles, wherein the hydrogel particles comprise PVA, wherein the hydrogel particles are injectable in size; b) at least one type of gellant, wherein the gellant is embedded within the hydrogel particles; and e) a hydrogel solution, wherein the hydrogel particles are suspended in the precursor hydrogel solution, and wherein the gellant diffuses out of the hydrogel particles when the composition is injected to a body cavity, thereby resulting in gelling of the surrounding hydrogel matrix.

In another aspect, the invention provides methods of making fabricated hydrogel compositions comprising: a) providing pre-gelled hydrogel particles; b) loading the hydrogen particles with at least one type of gellant; and c) providing a precursor hydrogel solution to suspend the hydrogel particles prior to application of the composition formed thereby.

In another aspect, the invention provides methods of making fabricated hydrogel compositions comprising; a) providing pre-gelled hydrogel particles, wherein the hydrogel particles comprise PVA, and wherein fee hydrogel particles are injectable in size; b) loading the hydrogen particles with at least one type of gellant; and c) providing a precursor hydrogel solution to suspend the hydrogel particles prior to application of the composition formed thereby.

In another aspect, the invention provides methods of making fabricated hydrogel compositions comprising: a) providing pre-gelled hydrogel particles, wherein the hydrogel particles comprise PVA, and wherein the hydrogel particles are injectable in size; b) providing a precursor hydrogel solution to suspend the hydrogel particles prior to application of the composition formed thereby; and c) loading the hydrogel solution with at least one type of gellant, thereby resulting in the gelling of the hydrogel solution, trapping the hydrogel particles in a continuous phase of gelled hydrogel solution.

In another aspect, the invention provides methods of implanting fabricated hydrogel particles into a selected site of a mammal to form a solid implant, wherein the method comprises: a) implanting a hydrogel composition into a selected site in a mammal, wherein the hydrogel composition comprises fabricated hydrogel particles, at least one type of gellant, wherein the gellant is embedded within the hydrogel particles, and a hydrogel solution in which the hydrogel particles are suspended in, thereby forming a hydrogel matrix; and b) allowing the gellant to diffuse out of the hydrogel particles, thereby gelling the surrounding of the hydrogel matrix and forming a solid implant.

In another aspect, the invention provides methods of implanting fabricated hydrogel particles into a selected site of a mammal to form a solid implant, wherein the method comprises: a) injecting a hydrogel composition into a selected body cavity in a mammal, wherein the hydrogel composition comprises fabricated injectable-sized hydrogel particles, at least one type of gellant, wherein the gellant is embedded within the hydrogel particles, and a hydrogel solution in which the hydrogel particles are suspended in thereby forming a hydrogel matrix; and b) allowing the gellant to diffuse out of the hydrogel particles into the body cavity, thereby gelling the surrounding of the hydrogel matrix in the body cavity and forming a solid implant.

According to another aspect of the invention, the hydrogel particles or a composition comprising the particles are implanted into a mammal in a surgical procedure for intervertebral disc replacement, wound care, cartilage replacement, joint replacement, implantation as a surgical barrier or a gastrointestinal device, a cosmetic and reconstructive operation, or breast or muscle enlargement.

According to another aspect of the invention, the hydrogel particles or a composition comprising the particles are implanted into a mammal to fill-in a cavity in a cartilage defect, in a joint such as hip, knee, or a nuclear cavity, the nuclear space within the intervertebral disc, and act as an articular or load-bearing surface.

In another aspect, the invention provides methods of treating a mammal comprising implanting fabricated hydrogel particles into a selected site of the mammal to form a solid implant, wherein the method comprises: a) implanting the fabricated hydrogel particles into the selected site of the mammal, wherein the fabricated hydrogel particles comprise at least one type of gellant, wherein the gellant is embedded within the hydrogel particles, and wherein the fabricated hydrogel particles are suspended in a precursor hydrogel solution, thereby forming a hydrogel matrix; and b) allowing the gellant to diffuse out of foe hydrogel particles, thereby gelling the surrounding of the hydrogel matrix and forming a solid implant.

In another aspect, the invention provides methods of treating a mammal comprising implanting fabricated hydrogel particles in a mammal to form a solid implant, wherein the method comprises: a) injecting the fabricated hydrogel particles into a selected body cavity of the mammal, wherein the fabricated hydrogel particles are injectable in size and comprise at least one type of gellant, wherein the gellant is embedded within the hydrogel particles, and wherein the fabricated hydrogel particles are suspended in a precursor hydrogel solution, thereby forming a hydrogel matrix; and b) allowing the gellant to diffuse out of the hydrogel particles into the body cavity, thereby gelling the surrounding of the hydrogel matrix in the body cavity and forming a solid implant.

According to another aspect of the invention, the mammal is treated for intervertebral disc replacement, wound care, cartilage replacement, joint replacement, implantation as a surgical barrier or a gastrointestinal device, a cosmetic and reconstructive operation, or breast or muscle enlargement.

According to another aspect of the invention, the mammal is treated for a cartilage defect, in a joint such as hip, knee, or a nuclear cavity, the nuclear space within the intervertebral disc, and act as an articular or load-bearing surface.

In another aspect, the invention provides kits for providing a hydrogel composition to a region of interest comprising: a container of pre-gelled or pre-solidified a hydrogel particles, wherein the hydrogel particles are loaded or embedded with at least one gellant; a container containing hydrogel solution; and a delivery device.

In another aspect, the invention provides methods of making crosslinked hydrogel particles comprising: a) forming an emulsion of a hydrogel solution in oil, thereby forming pre-solidified or pre-gelled hydrogel particles; b) cross-linking the hydrogel particles, thereby forming crosslinked hydrogel particles; and c) isolating the crosslinked hydrogel particles from the oil emulsion formed thereby.

In another aspect, the invention provides methods of making a crosslinked hydrogel composition comprising: a) forming an emulsion of a hydrogel solution in oil, thereby forming pre-solidified or pre-gelled hydrogel particles; b) cross-linking the hydrogel particles, thereby forming crosslinked hydrogel particles; e) isolating the crosslinked hydrogel particles from the oil emulsion; d) loading the crosslinked hydrogen particles with at least one type of gellant; and e) providing a precursor hydrogel solution to suspend the crosslinked hydrogel particles prior to application of the composition formed thereby.

In another aspect, the invention provides methods of making a crosslinked hydrogel composition comprising: a) forming an emulsion of a hydrogel solution in oil, thereby forming pre-solidified or pre-gelled hydrogel particles; b) cross-linking the hydrogel particles, thereby forming crosslinked hydrogel particles: c) isolating the crosslinked hydrogel particles from the oil emulsion, and wherein the hydrogel particles are injectable in size; d) loading the crosslinked hydrogen particles with at least one type of gellant; and e) providing a precursor hydrogel solution to suspend the crosslinked hydrogel particles prior to application of the composition formed thereby.

In another aspect, the invention provides methods of making a crosslinked hydrogel composition comprising: a) forming an emulsion of a hydrogel solution in oil, thereby forming pre-solidified or pre-gelled hydrogel particles; b) cross-linking the hydrogel particles, thereby forming crosslinked hydrogel particles; c) isolating the crosslinked hydrogel panicles from fee oil emulsion, wherein the hydrogel particles comprise PVA, and wherein the hydrogel particles are injectable in size; d) loading the crosslinked hydrogel particles with at least one type of gellant; and e) providing a precursor hydrogel solution to suspend the crosslinked hydrogel particles prior to application of fee composition formed thereby.

According to another aspect of the invention, the hydrogel particles are crosslinked by electron-beam radiation, gamma-radiation, beta-emitters, glutaraldehyde crosslinking, epichlorohydrin (EP) crosslinking, or by photo-initiated crosslinking.

According to another aspect of the invention, the fabricated hydrogeis comprise domains of different hydrogel particles.

According to another aspect of the invention, the PVA hydrogels comprise domains of polyacrylamide particles or PVA gel containing mechanically deformed hydrogel particles.

According to another aspect of the invention, the PVA hydrogels comprise oriented domains of hydrogel particles or domains of mechanically deformed hydrogel particles.

According to another aspect of the invention, the hydrogels comprise a polymer, polymer blends, or copolymers selected from fee group consisting of polyvinyl alcohol (PVA), polyvinyl pyrrolidone (PVP), alginates, polysaccharides, poly-N-isopropyl acrylamide (PNIAAm), or combinations of two or more thereof.

According to another aspect of the invention, the hydrogels comprise polyvinyl alcohol (PVA).

According to another aspect of the invention, the hydrogels comprise polyvinyl alcohol (PVA) copolymerized and/or blended with at least one of the other polymers.

According to another aspect of the invention, the gellant is selected from the group consisting of salts, alcohols, polyols, amino acids, sugars, proteins, polysaccharides, an aqueous solution thereof, or mixtures of two or more thereof.

According to another aspect of the invention, the gellant is selected from the group consisting of polyethylene glycol (PEG), poly ethylene oxide (PEO), polyvinyl pyrrolidone (PVP), poly-N-isopropyl acrylamide (PNIPAAm), chondroitin sulfate, dextran sulfate, dermatin sulfate and the like, or combinations of two or more thereof.

According to another aspect of the invention, the gellants comprise polyethylene glycol (PEG).

According to another aspect of the invention, the hydrogel solutions comprise polyvinyl alcohol (PVA), polyvinyl pyrrolidone (PVP), poly-N-isopropyl acrylamide (PNIAAm), or combinations of two or more thereof.

According to another aspect of the invention, the hydrogel solution is a polyvinyl alcohol (PVA) solution.

According to another aspect of the invention, the hydrogel particles are spherical, elliptical, or irregular in shape, or mixtures of two or more thereof.

According to another aspect of the invention, the hydrogel particles are agglomerations of a number of particles of a similar or different shapes.

According to one aspect of the invention, hydrogel particles used in medical implants or devices are fabricated by molding the hydrogel particles along with a matrix by mixing gellants with a hydrogel solution outside of a body. The mixing of gellants with the hydrogel solution and the hydrogel particles are carried out prior to implanting the device in the body. The gelation of the matrix also can occur by the gellants diffusion out of the hydrogel particles.

According to another aspect, medical implants or devices are fabricated by combining the hydrogel particles with a hydrogel matrix outside, of a body, molding, packaging, and sterilizing prior to shipping for use in humans. According to another aspect, the fabricated implant or device comprises domains of the same hydrogel. According to another aspect, the fabricated implant or device comprises domains of different hydrogels.

Unless otherwise defined, all technical and scientific terms used herein in their various grammatical forms have the same meaning as commonly understood by one of ordinary skill, in the art to which this invention belongs. Although methods and materials similar to those described herein can be used in the practice or testing of the present invention, the preferred methods and materials are described below. In case of conflict, the present specification, including definitions, will control. In addition, the materials, methods, and examples are illustrative only and are not limiting.

Further features, objects, and advantages of the present invention are apparent in the claims and the detailed description that follows. It should be understood, however, to that the detailed description and the specific examples, while indicating preferred aspects of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows PVA hydrogel particles prepared at room temperature as an emulsion in paraffin oil. Particles were stored in water for a month and blot-dried prior to photographing.

FIG. 2 shows PVA hydrogel particles prepared in emulsion with Vitamin E. Particles were stored in water for a month and blot-dried prior to photographing.

FIG. 3 shows PVA hydrogel particles prepared at room temperature emulsion from separate dispersion. Particles were stored in water for 2 weeks and blot-dried prior to photographing.

FIG. 4 shows collected PVA hydrogel particles in a beaker after PEG immersion treatment for 1 day. PEG-embedded or PEG-loaded PVA hydrogel particles were collected after draining the excess liquid.

FIG. 5 shows PEG-embedded or PEG-loaded hydrogel particles dispersed in 15% PVA solution.

FIG. 6 shows PEG-embedded or PEG-loaded hydrogel particles dispersed in 15% PVA solution after gelling at room temperature for 1 day.

FIG. 7 shows sintered gel particles in PVA matrix to form a new hydrogel. New hydrogels are formed from PEG-embedded or PEG-loaded hydrogel particles and surrounding matrix gelled through PEG diffusion from the particles.

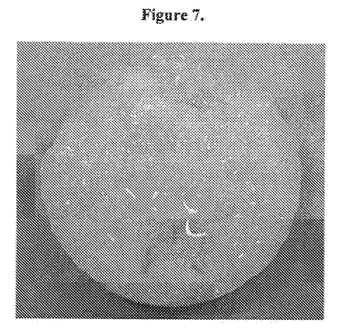

FIG. 8 is a schematic diagram of a hydrogel matrix containing domains of hydrogel particles.

FIG. 9 is a schematic diagram of a hydrogel matrix containing a mixture of domains of different types or different sizes of hydrogel particles.

FIG. 10 is a schematic diagram of a hydrogel matrix containing a mixture of domains of hydrogel particles of two different chemical structures.

FIG. 11 is a schematic diagram of a hydrogel matrix containing domains of oriented of mechanically deformed hydrogel particles.

FIG. 12 is a schematic diagram of a hydrogel matrix containing domains of differently oriented hydrogel particles.

FIG. 13 is a schematic diagram of a hydrogel matrix containing a mixture of domains of differently oriented hydrogel particles of two different chemical structures.

FIG. 14 is a schematic diagram of a hydrogel matrix containing a mixture of domains of different types, sizes, and orientations of hydrogel particles of two different chemical structures.

### DETAILED DESCRIPTION OF THE INVENTION

This invention provides fabrication of hydrogels and hydrogel particles. In some of the embodiments the hydrogel particles carry gellant and when the particles are mixed with a hydrogel solution, the gellant diffuses out of the particles and result in gelation of the surrounding hydrogel matrix. Alternatively, particles can be injected with liquid gel that already contains gellant.

According one embodiment of this disclosure, the pre-gelled or pre-solidified hydrogel particles are injected along with a precursor hydrogel solution. The pre-gelled or pre-solidified hydrogel particles are suspended in a liquid gel solution and injected into the body cavity. The precursor hydrogel solution forms a continuous matrix-containing the particulate solid gel particles in the cavity as the matrix gels in situ. This approach minimizes the amount of precursor hydrogel solution administered into the cavity since much of the volume can be pre-gelled particles. Therefore, inside the cavity, only a small proportion of the injected mass undergoes the gelation process.

One advantage of this inventive technique is that different kinds of hydrogel particles can be blended together and injected with different types of matrices. Another advantage is that if a gellant is needed to gel the surrounding matrix fluid, the pre-gelled or pre-solidified hydrogel particles can contain the gellant. Upon entering the body cavity, diffusion of the gellant out of the hydrogel particles initiates the gelation of the matrix. This type of particle-matrix can result in a heterogeneous hydrogel system with improved mechanical properties. Yet another advantage of using hydrogel particles is that the particulate gels can be modified by radiopacifers, such as barium sulfate or zirconia oxide, or nanoparticles, such as nanoclay, for example, laponite and montmorrilonite. The modification with nanoparticles can serve the purpose of improving the mechanical properties of the hydrogel-particles.

The pre-gelled or pre-solidified hydrogel particles can be loaded with biologically active molecules and/or pharmaceutically effective substances. Additionally, the particle size distribution and shape of the hydrogel particles can be controlled.

The hydrogel particles can be produced by forming an emulsion of the liquid gel system followed by gelation of the liquid gel system within the emulsion. The particles thus formed can be collected from the emulsion. Alternatively, an atomizer can be used to spray liquid droplets of the liquid gel into a container where gelation can occur. The container can be held at reduced pressure, and/or at temperatures greater or lower than room temperature. Atomization also can be performed using a room temperature solution into a body temperature container; the droplets thus formed by the atomization are heated up to the body temperature inside the container to obtain the gel particles (see for example, Lowman et al. US Publication No. 20040220296). Alternatively, the atomizer cars be used to spray-dry the liquid gel system, thus forcing gelation through dehydration. The hydrogel particles also can be obtained through conventional grinding or milling techniques to generate small hydrogel particles. The liquid gel used to generate the particles need not be water based.

According to one embodiment of this disclosure, gellant particle size ranges between about 0.1 μm and about 5.0 mm. The gellant particles can have a phase transition temperature greater than room temperature but less than or equal to body temperature. These particles cars be dispersed into the precursor hydrogel solution. The mixture can be injected into the body. The gellant particles can undergo the solid to liquid phase transition upon insertion into the body and release the gellant into the surrounding matrix, and thus begin the gelation process of the precursor hydrogel solution (see Ruberti and Braithwaite, US Published Document No. 20040171740),

Alternatively, the gellant particles can be prepared with a liquid core of the gellant material and a solid shell of a material that undergoes a phase transition at a temperature greater than room temperature but less than or equal to body temperature. Upon injection into the body, the solid shell melts, releasing the liquid gellant into the surrounding precursor hydrogel solution, inducing gelation in situ.

The term “gellant” refers to a gelling or solidifying agent. The gellants can be salts, alcohols, polyols, amino acids, sugars, proteins, polysaccharides, aqueous solutions thereof, and mixtures of two or more thereof. For example, the gellants used in loading hydrogel particles can be polyethylene glycol (PEG), polyethylene oxide (PEO), chondroitin sulfate, dextran sulfate, dermatin sulfate, and the like.

The terms “gelation” and “gelling” refer to a process of gel formation or gelation of a matrix containing hydrogel particles in presence of at least one gellant. These terms also refer to the formation of permanent physical cross-links due to the crystallization of the polymer solution, for example, the PVA solution, and/or the gellants, for example, the PEG, that are diffused out of the hydrogel particles. The terms gelation and gelling also refer to the formation of chemical crosslinks formed in the polymer solution, whereby the chemical crosslinks can be induced by radiation or by a chemical crosslinking agent such as glutaraldehyde or epichlorohydrin. Most polymer solution can be crosslinked to undergo gelation by ionizing radiation. PVA polymer solutions can be crosslinked by glutaraldehyde or epichlorohydrin to undergo gelation. The terms gelation and gelling also refer to phase transformation of the polymer solutions such as PNIAAm solutions. The terms also refer to the formation of ionic interactions that act as physical crosslinks and cause gelation.

The term “precursor hydrogel solution” refers to any solution or mixture of solutions that is capable of undergoing gelation. The gelation process can occur through various association or interactions, for example, through the development of physical associations, such as crystalline junctions, ionic interactions, or crosslinks, hydrogen bonding or hydrophilic associations.

The surfaces of the particles can be treated to decrease the rate of diffusion of the gellants out of the particles into the matrix, so as to control the kinetics of gelation of the surrounding matrix.

The hydrogel particles can be of various shapes, preferably spherical, and are embedded or loaded with one or more gellant types.

The hydrogel particles can be of various sizes. The hydrogel particles can be small enough in size to pass through an injection needle. Size of the needle can vary, for example, a needle size of about 33, about 28, about 25, about 22, about 20, or about 18 gauge or lower, or any size thereabout or therebetween, is preferred. The inner diameter of the needle also can vary, for example, an inner diameter of about 0.025 mm or more, about 0.089 mm or about 0.10 mm or more, or any diameter thereabout or therebetween.

The term “injectable in size” refers to a size of a hydrogel particle that is of a size that can pass through an injection needle, as described above.

Hydrogels generally include polymer, polymer blends, or copolymers of polyvinyl alcohol (PVA), polyvinyl pyrrolidone (PVP), polyacrylamide (PAAM), Polyacrylic acid (PAA), alginates, polysaccharides, polyoxyethylene-polyoxypropylene co-polymers, poly-N-alkylacrylamides, or poly-N-isopropyl acrylamide (PNIAAm).

The hydrogel particles can comprise polyvinyl alcohol (PVA) copolymerized and/or blended with at least one of the other polymers or gellants, for example, polyvinyl pyrrolidone (PVP), poly-N-isopropyl acrylamide (PNIPAAm), or combinations of two or more thereof.

The hydrogel particles can have orientation induced by mechanical deformation, such as one induced by uniaxial deformation. The hydrogel particles can be radiation crosslinked after they are fabricated. The hydrogel particles can be hydrated or dehydrated.

In one aspect of the invention, the hydrogel particles comprise polyvinyl alcohol (PVA), fabricated and loaded with a gellant, such as polyethylene glycol (PEG). The particles are fabricated and embedded with PEG molecules. The molecular weight of the PEG is varied to control the diffusion rate of PEG out of the particles, and the gelling activity of the PEG, when the particles are mixed with a hydrogel solution.

In one aspect of the invention, the hydrogel particles are fabricated and subsequently mechanically deformed. The mechanical deformation generates permanent deformation causing molecular orientation within the particles; for example, if the particles are deformed under uniaxial compression there is biaxial orientation of the molecules in the particles resulting m anisotropy within the particles, that is different mechanical properties in different directions. The mechanically deformed particles are loaded with a gellant such as polyethylene glycol (PEG). The molecular weight of the PEG is varied to control the diffusion rate of PEG out of the particles, and the gelling activity of the PEG when the particles are mixed with a hydrogel solution.

In another aspect of the invention, the hydrogel particles are fabricated and subsequently mechanically deformed. The mechanically deformed particles are placed in a hydrogel solution and the surrounding solution is gelled; thus forming a hydrogel containing mechanically deformed particles. In one embodiment, the deformed particles are randomly oriented in the matrix. In another embodiment, the deformed particles have a preferred orientation, for example, the orientation of the deformed particles is obtained by using a flow field in the hydrogel solution; or alternatively, the gelled hydrogel containing deformed hydrogel particles is mechanically deformed.

The term “Mechanical deformation” includes uniaxial, channel flow, uniaxial compression, biaxial compression, oscillatory compression, tension, uniaxial tension, biaxial tension, ultra-sonic oscillation, bending, plane stress compression (channel die) or combinations of any of the above. The deformation could be static or dynamic. The dynamic deformation can be combinations of the deformation modes in small or large amplitude oscillatory fashion. Ultrasonic frequencies can be used.

In another embodiment, the hydrogel particles are dehydrated before mixing with the hydrogel solution. The dehydration is carried out by heating the hydrogel particles, by placing them in vacuum, by placing them in vacuum at room temperature, by placing them in vacuum at an elevated temperature such as 40° C. or 100° C., by placing them in a solution that has higher affinity to water than the hydrogel particles for instance placing PVA particles in 100% PEG or an aqueous PEG solution.

In another embodiment, the dehydrated hydrogel particles are radiation crosslinked.

In another embodiment, the dehydrated hydrogel particles are rehydrated by immersion in water or saline solution. The rehydration can be carried out at different temperatures, ranging from a temperature below the room temperature to a temperature above 160° C. For example, the rehydration can be carried out at about 5° C., about 10° C., about 20° C., about 30° C., about 40° C., about 50° C., about 60° C., about 70° C., about 80° C., about 100° C. or at any temperature thereabout or therebetween. The rehydration also can be carried out under atmospheric pressure at about 120° C., about 140° C., about 160° C., or about 200° C., or at any temperature thereabout or therebetween. The rehydration is carried out under about 1 atm (atmospheric) to about 200 atm pressure, when the temperature is above about 100° C. For example, the atmospheric pressure is about 4 atm, 5 atm, 10 atm, 15 atm, 50 atm, 100 atm, 150 atm, or 180 atm, or under any pressure thereabout or therebetween.

In another embodiment, the PEG loaded PVA hydrogel particles are mixed with an aqueous solution of PVA. As the PEG molecules slowly diffuse out of the particles, they initiate the gelation of the surrounding PVA solution. This process leads to the formation of a PVA hydrogel matrix embedded with the previously gelled PVA hydrogel particles.

According to one aspect of the invention, the hydrogel particles are fabricated and loaded with a gellant such as polyethylene glycol (PEG), by soaking the gel particles in a water/PEG mixture to control the extent of PVA hydrogel particle shrinkage when immersed in the PEG. The gelled and initially hydrated PVA hydrogel particles lose water, i.e. dehydrate, when immersed in 100% PEG. The dehydration causes shrinkage and results in smaller PVA hydrogel particles. To limit the extent of shrinkage of the hydrogel particles, soaking in a water/PEG mixture, instead of 100% PEG, is used to load PEG into hydrogel particles. The ratios of the water/PEG mixture are varied to control the final particle size and the amount of PEG loaded into the particles.

According to another aspect of the invention, the hydrated and gelled. PVA hydrogel particles are loaded with PEG by immersion in a PEG or PEG/water mixture forming a suspension. The suspension is gamma-radiation sterilized prior to shipping for use in patients.

According to another aspect of the invention, the PEG-loaded PVA hydrogel particles are mixed with an aqueous solution of PVA. As the PEG molecules slowly diffuse out of the particles, they initiate the gelation of the surrounding PVA solution. This process leads to the formation of a PVA hydrogel matrix embedded with the previously gelled PVA hydrogel spheres.

According to one aspect of the invention, one method of forming the PVA hydrogel particles is by mixing a 10% PVA aqueous solution with 40% PEG at an elevated temperature (for example, at 95° C.), The PVA/PEG blend is then mixed with an oily substance (such as mineral oil or paraffin oil) and the oil-PVA/PEG mixture is violently stirred to form an emulsion at an elevated temperature. The emulsion is then cooled down to room temperature to cause the gelation of the spherical phases of the PVA/PEG mixture. Thus, pre-gelled or pre-solidified PVA hydrogel particles are formed.

In another aspect of the invention, one method of forming the PVA hydrogel spheres is by making a 10% PVA aqueous solution at an elevated temperature (for example, up to about 95° C.) or at room temperature. The PVA solution is then mixed with an oily substance (such as mineral oil or paraffin oil) and the oil-PVA mixture is stirred to form an emulsion at an elevated temperature or at room temperature. This emulsion is then irradiated to crosslink the PVA particles at an elevated temperature (for example, up to about 95° C.) and then cooled down to room temperature. Alternatively, to the irradiation is carried out at room temperature.

The particle size and shape are controlled by varying the molecular weight of the paraffin oil, the peak temperature at which the emulsion is formed, the rotational speed of stirring, the emulsion concentration and/or the concentration of the aqueous PVA solution.

The small particles of hydrogel, for example, in the shape of spheres, are used as a carrier of gellant. The gellant carrying particles are mixed with the hydrogel solution to cause the gelation of the surrounding hydrogel matrix. This way, only a small portion of the hydrogel solution is necessary to be gelled either in situ or in the operating theatre to cast a desired shape.

The hydrogel particles, according to the invention, can be of any shape or mixtures of two or more thereof, for example, a spherical shape, an elliptical shape, and/or irregular shapes. They also can be agglomerations of a number of particles of similar or different shapes.

In another aspect, the invention provides methods for early treatment of joint disease by providing a hydrogel cushion formed in situ between load-bearing surfaces in the joint. For example, a hydrogel cushion is formed in situ within the hip joint by dislocating the head of the femur, filling the exposed cavity within the joint with a hydrogel solution containing hydrogel particles with embedded or loaded gellants, replacing the head of the femur, and allowing the gellants to diffuse out of the hydrogel particles to initiate the gelation process in situ. The same is done with other joints such as knee, shoulder, elbow, and the like.

According to another aspect of the invention, a paste of gellant loaded hydrogel particles are mixed with a solution of the same or different type of hydrogel. The paste is a viscous substance and is used to fill a cavity within a mammalian body, for example, a human body. The cavities could be the nuclear cavity in the intervertebral disc, a cartilage defect in a joint such as the hip or knee, and the like.

The hydrogel particles or a composition comprising the hydrogel particles can be considered or used in a variety of biomedical applications, for example, intervertebral disc replacement or disc augmentation, wound care, cartilage replacement, joint replacement, surgical barriers, gastrointestinal devices, drug delivery, cosmetic and reconstructive surgery (such as nose, ear, or chin and the like), and breast enlargement.

The hydrogel particles or a composition comprising the particles can be. implanted into a mammal in a surgical procedure for intervertebral disc replacement wound care, cartilage replacement, joint replacement, implantation as a surgical barrier or a gastrointestinal device, a cosmetic and reconstructive operation, or breast or muscle enlargement.

The pre-gelled hydrogel particles also can be loaded with biologically active molecules or pharmaceutically effective substances, such as drugs, for local delivery. For example, antibiotics to prevent infection at and around the surgical site. By loading the hydrogel particles and not the matrix with the active molecules or substance, a virtually constant elution profile can be obtained.

According to another aspect of the invention, the paste of the pre-gelled hydrogel particles loaded with a gellant and/or other molecules are mixed with an aqueous solution of the hydrogel particles and are applied to a cartilage defect during surgery. The kinetics of gelation for the surrounding aqueous hydrogel solution are tailored to achieve gelled structure within a reasonable time period to fill-in the cartilage defect and act as an articular, load-bearing surface. This is applicable for patients with early arthritic cartilage lesions and/or patients with cartilage lesions induced by trauma.

According to another aspect of the invention, the body cavity, such as the cartilage defect in a hip or knee, or the nuclear space within the intervertebral disc, are cooled down prior to filling the detect with the pre-gelled hydrogel particles, gellant, and/or hydrogel solution. Cooling of the cartilage cavity is achieved by flushing the cavity with saline solution. The lower local temperature accelerates the gelling kinetics of the hydrogel solution that acts as the matrix to the hydrogel particles.

According to another aspect of the invention, the pre-gelled hydrogel particles are loaded by the gellant and the particles are subsequently treated to form an outer skin layer to slow down the diffusion of the gellant out of the particles when the particles are immersed in the hydrogel solution. The treatment can be a thermal treatment or can be immersion in a concentrated gellant, such as 100% PEG, to slightly dehydrate the outer layer of the hydrogel particles. The treatment results in a stiffer matrix with locally reduced water content and slower diffusion characteristics of the outer layer of the pre-gelled hydrogel particles.

According to another aspect of the invention, the pre-gelled hydrogel particles are formed by forming an emulsion of a hydrogel aqueous solution in oil and irradiating the emulsion by electron-beam radiation or gamma-radiation. The radiation dose is at least about 1 kGy, for example, about 25 kGy, between 25 and 1000 kGy, about 50 kGy, about 100 kGy, and about 150 kGy. The irradiation crosslinks the hydrogel particles in the small domains of the emulsion and forms crosslinked gel. Following irradiation, the hydrogel particles are removed from the oil emulsion and are then used.

Yet, according to another aspect, the pre-gelled hydrogel particles are formed by forming an emulsion of a hydrogel aqueous solution in oil and crosslinking the hydrogel molecules by photo-reactive chemical crosslinkers, thereby forming a crosslinked gel. Following cross-linking, the particles are removed or isolated from the oil emulsion and are then used, or are further soaked in a gellant to load, for example, acrylamide monomer or dimethyl acrylamide monomer is dissolved in water with N,N′-methylenebisacrylamide crosslinker and oxo-gluteric acid. This mixture is then placed in oil to form an emulsion and the emulsion is then exposed to UV light or the like for crosslinking. Alternatively, the mixture also can contain PVA prior to UV crosslinking.

In one aspect of the invention, the pre-gelled hydrogel particles are stored in a solution of the gellant or pure gellant, such as a low molecular weight polyethylene-glycol.

In another aspect, the pre-gelled hydrogel particles prepared by using radiation are loaded by gellant and/or drugs and/or other molecules. These gellant-loaded particles of the hydrogel are then used to gel the surrounding precursor hydrogel solution matrix.

In one aspect, the hydrogel particles are loaded with the gellant by placing the particles and the gellant in supercritical fluid, such as carbon dioxide, at about 40° C. and at least 1100 psi of carbon dioxide gas. The supercritical fluid increases the diffusion of the gellant into the hydrogel particles.

“Supercritical fluid” refers to what is known in the art, for example, supercritical propane, acetylene, carbon dioxide (CO₂) (see, for example, U.S. Pat. No. 6,448,315 and WO 02/26464).

Advantage of forming the hydrogel particles is the ability to modify their structure by the addition of nanometer-sized particles. In one aspect, for instance, the hydrogel solutions are loaded with clay (such as montmorrilonite or laponite). The clay-loaded hydrogel solution at an elevated temperature is blended with a gellant and put into an emulsion. Upon cooling down to room temperature, the hydrogel emulsion droplets form gel particles containing clay. Similarly, the clay-loaded hydrogel solution is put into an emulsion without the gellant and the emulsion is irradiated using electron-beam, gamma- or beta-emitters to form crosslinked hydrogel particles containing clay. The radiation dose is at least about 1 kGy, for example, about 25 kGy, between 25 and 1000 kGy, about 50 kGy, about 100 kGy, or about 150 kGy. The hydrogel particles thus prepared are then loaded with a gellant and used in the gelation of the surrounding hydrogel solution matrix.

According to another aspect of the invention, the pre-gelled hydrogel particles are formed by using a freeze-thaw technique. An emulsion of the hydrogel solution in oil is prepared, the emulsion is cooled down to below 0° C., and then heated back to room temperature. The number of freeze-thaw cycles can increase the stiffness of the hydrogel particles thus formed. The freeze-thaw method cars be used with PVA solution; specifically a PVA aqueous solution (for example 10%) is prepared and mixed with the oil. An emulsion is formed by stirring and/or shaking the mixture. The stirred and/or shaken emulsion is then placed is a freezer. The stirring or shaking is continued to maintain the emulsion and prevent coalescence of the hydrogel particles. The emulsion is then removed from the freezer for thawing. The gelled hydrogel particles thus formed also can be used in the embodiments described herein.

The size distribution of the hydrogel particles is varied to serve a specific purpose. For example, for implanting a hydrogel formulation by injection methods, hydrogel particles are injectable in size. However, for direct implantation of a hydrogel formulation, particle sizes can be larger, if desired.

Hydrogel particles prepared by various methods, such as the emulsion/gellant, emulsion/freeze-thaw, or emulsion irradiation, can be blended together to form a mixture.

In all of the above described emulsions, a surfactant can be used to stabilize the emulsion and prevent coalescence of the hydrogel particles. The surfactant can be a detergent such as Pluronic® (polypropylene oxide-ethylene oxide) block copolymer), Tween 80 (polyoxyethylene sorbitan monooleate), and Span 80 (sorbitan monooleate), or a fatty acid such as sodium lauryl sulfate, sodium caprylate, and the like.

According to another aspect of the invention, the PVA hydrogel particles formed in the emulsion are crosslinked by adding epichlorohydrin (EP). The EP is added to the PVA solution before preparing the emulsion. Alternatively, the EP is added to the emulsion of the aqueous PVA. In one aspect, the PVA is pre-crosslinked with EP by mixing an aqueous PVA solution with EP and stirring at 50° C. in the presence of sodium hydroxide. The pre-crosslinked PVA solution is then put in an emulsion at 50° C. Vigorous stirring is maintained until the PVA particles are completely crosslinked.

According to another aspect of the invention, the gel is prepared with a small fraction of an added antioxidant or free radical scavenger, such as one of the tocopherols like vitamin E (α-tocopherol) or other tocopherol types, for example, β-, δ-, and γ-tocopherol. The gel containing the vitamin E is gamma sterilized. Vitamin E prevents or reduces the extent of crosslinking of the gel during the gamma sterilization. As a result, the sterilized gel can later be melted to form a gel solution. The melting of the gamma sterilized gel can be carried out in an operating theater to form a gel solution. Vitamin-E also acts as an antioxidant protecting the hydrogel against oxidation.

Polyvinyl alcohol) aqueous solution is prepared with PEG at an elevated temperature. The mixture is placed in a gamma sterilizable container and cooled down to room temperature. Upon cooling down, the PVA gel is formed with the PEG and possibly some excess liquid composed of water and PEG. This mixture can also be prepared with vitamin E in either the PVA solution or the PEG, so that there is vitamin E in the final gel form. The container that contains the PVA gel with the PEG and some excess liquid along with vitamin E is sealed and gamma sterilized. In the operating room, the container is heated to above the gel solution temperature (for example, above 70° C., preferably about 90 to about 95° C.). At this elevated temperature the gel is dissolved, and later is injected into a cavity in the human or animal body. The solution contains the gellant PEG; therefore re-gelation can take place inside this cavity. This example shows how a gel can be prepared with an antioxidant such as vitamin E so that it cars be gamma sterilized without crosslinking so that it can be melted later during surgery and injected into a body cavity.

According to another aspect of the invention, the hydrogel particles are generated by atomizing the hydrogel solution into a gellant-containing chamber. The solution of the hydrogel is prepared and placed in an atomizer. The atomizer can be used either at room temperature or at an elevated temperature, and the latter reduces the viscosity of the solution and improves the atomization. The atomization is then carried into a container where the gellant is present. The atomized particles of the hydrogel solution solidify or gel upon contacting the gellant inside the container. The microspheres of the hydrogel thus formed are collected from the container.

According to another aspect of the invention, the hydrogel solution and the gellant are prepared at an elevated temperature and placed in an atomizer. Before being placed in the atomizer, the mixture is kept above the temperature where gelling normally starts. For example, in the case of PVA/PEG mixture, the solution is kept at 90° C. The mixture of the hydrogel solution and the gellant is atomized at the elevated temperature into the container. The microspheres formed during the atomization rapidly cool down before reaching the bottom of the container and thus gel. In another aspect of this example, the container into which the hydrogel solution and gellant mixture is atomized, can contain further gellant to help the gelation of the microspheres. In other aspects, a lower concentration of the hydrogel solution can be used such as 2% PVA, to further reduce the viscosity of the hydrogel solution and improve the efficiency of the atomization process (that is to reduce the particle size formed). When lower concentrations of the solutions are used during the atomization, the gels formed may not be as strong. The strength of the gels can be increased by decreasing the amount of water present in the spheres through steps of drying (dehydration) and/or by further gelling by placing in a bath of gellant. In another aspect, the collection container can be held at reduced pressures to aid in the gelation process.

In another aspect of the invention, the microparticles of the hydrogel formed are used to carry a gellant to cause gelation in a hydrogel solution when the particles are placed in the hydrogel solution. When the hydrogel solution is gelled, it becomes a matrix containing the microparticles of the hydrogel, which also is referred to as a domain of hydrogel particles in the hydrogel matrix. In other aspects, the microparticles of the hydrogel carry nanoscale clay particles such as Laponite or Montmorrilonite; but the matrix does not. The result is a composite material with a gelled hydrogel matrix embedded with domains of hydrogel containing nanoscale particles, including clay. The particle size, the interparticle distance, and/or the concentration of the particles can be altered to manipulate certain properties of the hydrogels. In addition the relative concentrations of PVA in the matrix and in the particles also can be manipulated to modify the composite properties. Even when the particles do not contain other ingredients such as clay, the pre-gelled hydrogel particles with the surrounding hydrogel matrix can still result in a heterogeneous hydrogel system.

In another aspect, already formed hydrogel pieces are broken down by mechanical attrition. The attrition can be carried out in a freezer mill or by any other suitable technique to form the hydrogel particles.

According to another aspect of the invention, the microparticles of the hydrogel formed are reduced in size by further gelation by dehydration. The dehydrated smaller microspheres are injected into a body cavity where they re-swell, larger than the inlet hole, thus providing space filling support for that cavity.

Yet, according to another aspect of the invention, the hydrogel solution is injected directly into a pore gellant (or a solution of gellant) through a large gauge syringe needle or a small diameter orifice, in order to form strands of gelled hydrogel. These strands or filaments can be used as carriers of gellant as described above; and when placed in a hydrogel solution can cause the gelation of the surrounding hydrogel solution. The strands or filaments can be embedded in this hydrogel matrix. The strands or filaments can act as modifiers of the hydrogel matrix.

In one aspect, the microparticles of the hydrogel are formed with the PVA solution, are emulsified into a medium and are cooled down below room temperature, preferably below 20° C., more preferably below 10° C., more preferably about 7° C. The gellant is added to the emulsion PVA solution mixture before or after cooling down.

In another aspect of the invention, particles of pure gellant, or combinations of gellants are prepared that have a phase transition temperature greater than room temperature but less than or equal to body temperature. The activity of such gellant particles at room temperature is very low, however, increases upon injection into a body cavity. When the particles are placed in a precursor hydrogel solution, they do not induce gelation at room temperature storage and the gelation process begins when the particles are exposed to an elevated temperature inside the body. The gellant particles size ranges from about 0.1 micrometer to about 5.0 millimeters. The particles can be made by using processes described in embodiments of this disclosure or by using similar technologies known in the art.

In another aspect, of the invention, composite gellant particles can be prepared, which contain a liquid core of the gellants, and a solid shell of a material that has a phase transition temperature greater than room temperature but less than or equal to body temperature. These composite particles are blended into a precursor hydrogel solution. Under room temperature storage, the solid shell prevents the liquid core gellants from inducing associative gelation of the precursor hydrogel. After injection into a body at or about 37° C., the solid shell melts and release the gellants, which causes gelation of the precursor hydrogel solution. The composite particles size ranges from about 0.1 mm to about 5.0 μm. The particles can be made by using processes described in embodiments of this disclosure or by using similar technologies known in the art.

In one embodiment, fabricated articles are made using hydrogel particles and hydrogel solution; for example, hydrogel particles of PVA are mixed with a PVA solution and the solution is gelled using a gellant, radiation, or freeze-thaw.

The fabricated articles can be finished implants, preforms, machined into finished medical implants (such as plugs to fill cartilage defects, interpositional devices to act as a cushion in a joint), and tissue augmentation devices (such as breast implants and the like). The fabrication can be done by molding the hydrogel solution and hydrogel particles together in a mold. The mold dimensions can be tailored to account for shrinkage during gelation and swelling during subsequent rehydration. Other dimensional changes that may result from other steps, such as sterilization, dehydration, rehydration, mechanical deformation and the like, also can be accounted for in the mold dimensions so that the equilibrium device dimensions are those of the desired implant. Additionally, further machining also can be carried out.

The injectable hydrogel formulations, hydrogel solutions, hydrogel particles, mixtures of hydrogel solution and hydrogel particles, fabricated articles, implants, and the like, are packaged and sterilized before sending out for use in a mammalian body, such as a human body.

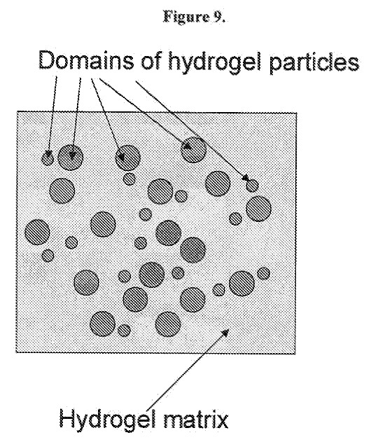

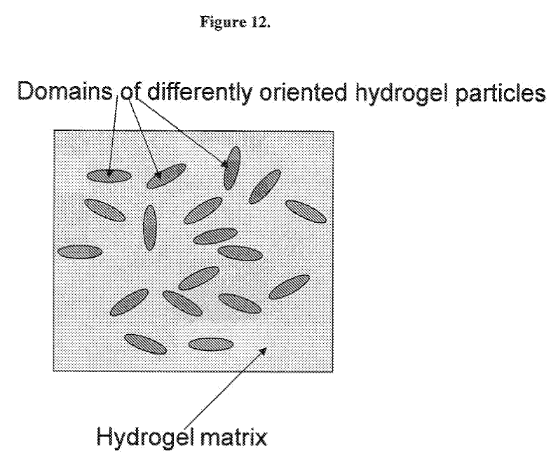

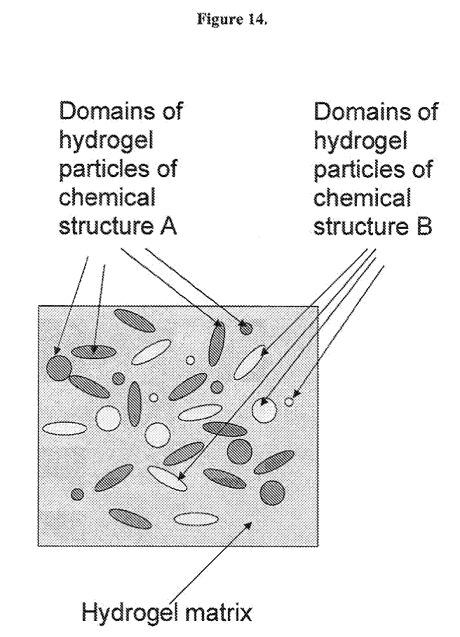

The term “domain” refers to hydrogel particles, clusters, or mixtures of hydrogel particles embedded or integrated in a hydrogel, thereby forming a hydrogel matrix containing domains of hydrogel particles. A domain of hydrogel particles is surrounded by or integrated with hydrogel that forms the hydrogel matrix. A domain of a hydrogel particle in a hydrogel matrix is the location where the particle is embedded into the matrix with or without any physical boundary between the particle and the matrix. A domain of hydrogel particles and the hydrogel matrix can have the same or different chemical structure or nature. (See FIGS. 9-14 for schematic diagrams of hydrogel matrices containing domains of hydrogel particles). A hydrogel matrix can comprise to multiple domains of different hydrogel particles (see FIGS. 9, 10, 13, and 14). Multiple domains can be in contact and in most embodiments the interstices of the domains are hydrogel matrix. In some embodiments, the interstices also can be filled with smaller domains of hydrogel particles (see FIGS. 9 and 14).

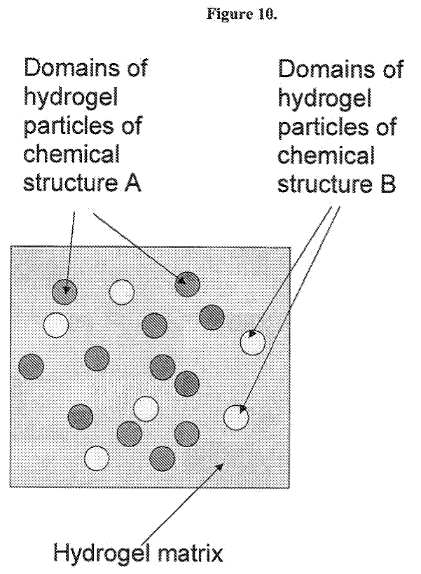

“An oriented domain” refers to domains having hydrogel particles having molecular orientation induced by mechanical deformation, or other forces or production methods, for example, vibration, ultra-sound, microwave, magnetic field, and the like. A domain of mechanically deformed hydrogel particles also can be referred to as an oriented domain. See FIGS. 11, 10, 13, and 14 for schematic diagrams of hydrogel matrices containing mechanically deformed and/or oriented domains of hydrogel particles.

In one embodiment, a fabricated hydrogel comprises domains of different types of hydrogel particles, for example, a PVA hydrogel matrix containing polyacrylamide particles or a PVA gel containing mechanically deformed hydrogel particles. The hydrogel particles comprise at least one type of gellant, wherein the gellant is embedded within the hydrogel particle, and wherein the gellant can diffuse out of the hydrogel particle, thereby gelling the surrounding hydrogel matrix. In another embodiment, a hydrogel matrix contains oriented domains of hydrogel particles.

According to another embodiment, medical implants or devices are fabricated by molding hydrogel particles along with a matrix, causing gelation of the matrix by mixing gellants with a hydrogel solution and the hydrogel particles at an elevated temperature, and by cooling the mixture to room temperature. In one aspect, the hydrogel particles are not injected into the body cavity directly, but are molded with the matrix outside of the body, allowing the matrix to gel or solidify by the gellants that are diffused out of the hydrogel particles, and/or by mixing gellants with a hydrogel solution and the hydrogel particles at an elevated temperature (for example, at about 90°C.). The mixture is cooled to room temperature to accelerate the gelation of the matrix.

According to another embodiment, medical implants or devices comprise hydrogel particles are fabricated and/or molded outside the body, and are packaged, sterilized, and shipped for use in humans.

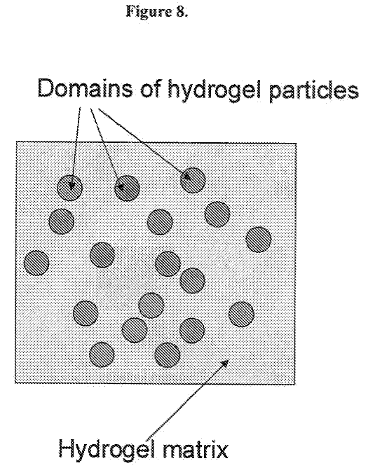

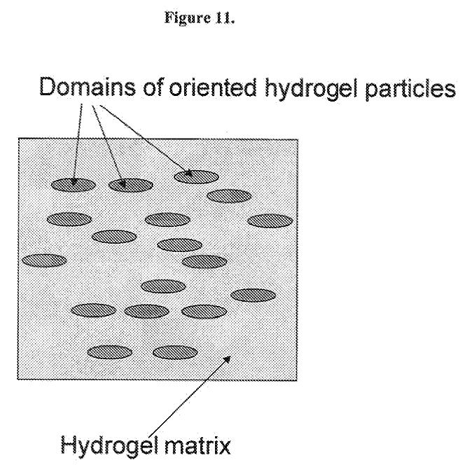

According to another embodiment, fabricated articles comprise hydrogel particles, wherein the fabricated articles comprise domains of the same type of hydrogel material (see FIGS. 8 and 11, for example).

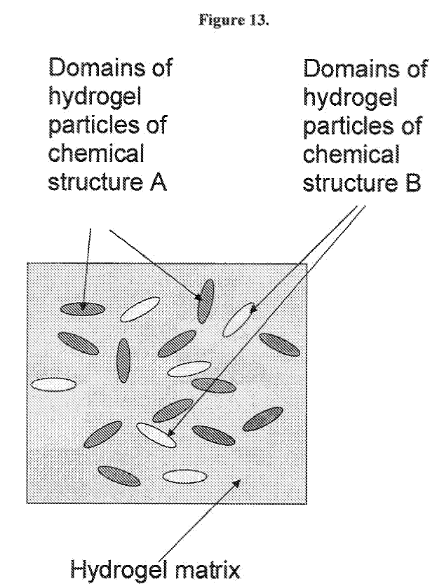

According to another embodiment, fabricated articles comprise hydrogel particles, wherein the fabricated articles comprise domains of different hydrogels (see FIGS. 10 and 13, for example). The difference in hydrogels particles forming domains in a hydrogel matrix can be in terms of size, shape, content, chemical structure or constituency (see FIG. 14, for example).

According to another embodiment, fabricated articles comprise hydrogel particles, wherein matrix of the fabricated articles comprise domains of hydrogels particles of two or more types and/or having two or more types of chemical structures (see FIG. 14, for example).

The invention is further described by the following examples, which do not limit the invention in any manner.

### EXAMPLES

### Example 1

### Room Temperature Emulsion in Paraffin Oil

Twenty grains of polyvinyl alcohol (PVA, MW=118,000) were added to 180 g of deionized water and stirred while heating for about 2 hours to prepare a fully dissolved 10% (wt) PVA solution. The dissolved PVA solution was kept in an air convection oven (DKN600, Yamato) at 90° C. for about 16 hours. Polyethylene glycol (PEG, MW=400) was heated to 90° C. in an air convection oven.

One hundred milliliters of light paraffin oil were placed in a 500 ml three-neck round bottom flask at room temperature. Then, two grams (2% w/v) of Tween 80 (polyoxyethylene-20-sorbitan monooleate) were added to the flask containing the paraffin oil and the mixture was stirred at 350 rpm. The stirring speed was maintained at 350 rpm throughout the procedure. Fifty grams of the previously prepared 10% (wt) PVA solution (at 90° C.) were slowly poured into the flask containing the above mixture and continued stirring at room temperature for at least 1 minute to form an emulsion. Subsequently, 30 grams of the PEG (at 90° C.) were added to the emulsion while stirring. The mixture was then stirred at room temperature for at least 30 minutes to ensure the gelation of the PVA domains.

Gel particles were collected from the emulsion mixture and then briefly washed with Xylene once and repeatedly washed with copious amounts of water.

Washed gel particles were stored in various ways, such as drying in an air convection oven at 40° C., storing in PEG, air, water, or saline at room temperature. Gel particles were well dispersed both in water and saline with no aggregation. The particles placed in PEG showed some de-swelling.

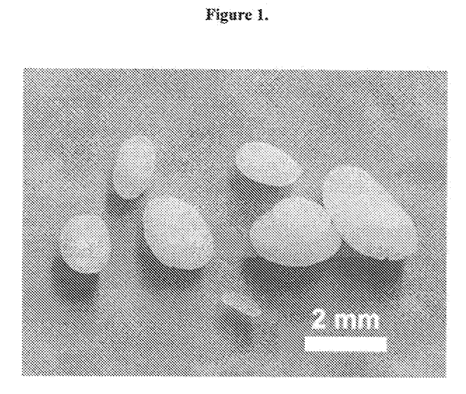

Particle sizes were about 0.8 to about 3.5 mm, and the shape varied from spheres to rod-like (see FIG. 1).

### Example 2

### Emulsion in Vitamin E

Twenty grams of polyvinyl alcohol (PVA, MW=118,000) were added to 180 grams of deionized water and stirred while heating for about 2 hours to prepare a folly dissolved 10% (wt) PVA solution. The dissolved PVA solution was kept in an air convection oven (DKN600, Yamato) at 90° C. for about 16 hours. Polyethylene glycol (PEG, MW=400) was heated to 90° C. in an air convection oven.

One hundred milliliters of vitamin E were placed in a 500 ml three-neck round bottom flask and heated. Then, 4 grains of Tween 80 (polyoxyethylene-20-sorbitan monooleate) were added to the flask containing vitamin E and the mixture was stirred at 500 rpm. The stirring speed was maintained at 500 rpm throughout the procedure. Fifty grams of the previously prepared 10% (wt) PVA solution (at 90° C.) were slowly poured into the flask above, while stirring on heat for at least 1 minute. Subsequently, 30 grams of the PEG were (at 90° C.) added to the emulsion while stirring. The mixture was then slowly cooled down to room temperature for at least 1 hour while stirring, to ensure the gelation of the PVA domains.

Gel particles were collected from the emulsion mixture and then washed with copious amounts of ethanol.

Washed gel particles were stored in various ways, such as drying in an air convection oven at 40° C., storing in PEG, air, water, or saline at room temperature. Gel particles were well dispersed both in water and saline with no aggregation. Particles obtained slightly yellow color, due to residual vitamin E, which was removed after immersion in ethanol for 1-2 days.

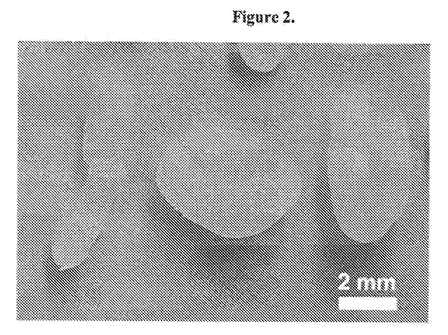

Particle sizes were 3-5 mm and shape was mostly oblong as shown in FIG. 2. The shape of the particles is changed to spherical by increasing the shear on the emulsion by increasing the rpm speed of the stirring of the emulsion.

### Example 3

### Room Temperature Emulsion in Paraffin Oil, PVA Injected

Twenty grams of polyvinyl alcohol (PVA, MW=118,000) were added to 180 grams of deionized water and stirred while heating for about 2 hours to prepare a fully dissolved 10% (wt) PVA solution. The dissolved PVA solution was kept in an air convection oven (DKN600, Yamato) at 90° C. for about 16 hours. Polyethylene glycol (PEG, MW=400) was healed to 90° C. in an air convection oven.

Disposable plastic syringes (10 ml) and hypodermic needles (19 gauge, stainless steel) were heated to 90° C. in an air convection oven.

One hundred milliliters of light paraffin oil were placed in a 500 ml three-neck round bottom flask at room temperature. Then 2 grams (2% w/v) of Tween 80 (polyoxyethylene-20-sorbitan monooleate) were added to the flask containing paraffin oil and the mixture was stirred at 350 rpm. The stirring speed was maintained at 350 rpm throughout the procedure.

Thirty grams of the previously prepared 10% (wt) PVA solution (at 90° C.) were poured into the plastic syringes and injected through the needle into the flask above, while stirring at room temperature to form an emulsion. Subsequently, 18 grams of the PEG (at 90° C.) were added to the emulsion while stirring. The mixture was then stirred at room temperature for at least 30 minutes to ensure the gelation of the PVA domains.

Gel particles were collected from the emulsion mixture and briefly washed with hexane and then repeatedly washed with copious amounts of water.

Washed gel particles were stored in various ways, such as drying in an air convection oven at 40° C., storing in PEG, air, water, or saline at room temperature.

### Example 4

### Room Temperature Emulsion From Two Separate Emulsions

Twenty grams of polyvinyl alcohol (PVA, MW=118,000) were added to 180 grams of deionized water and stirred while heating for about 2 hours to prepare a folly dissolved 10% (wt) PVA solution. The dissolved PVA solution was kept in an air convection oven (DKN600, Yamato) at 90° C. for about 16 hours. Polyethylene glycol (PEG, MW=400) was heated to 90° C. in an air convection oven.

One hundred milliliters of light paraffin oil were placed in a 600 ml beaker at room temperature. Then 2 grams of Tween 80 (polyoxyethylene-20-sorbitan monooleate) were added to the beaker and the mixture was stirred at 500 rpm. Thirty grams of the previously prepared 10% (wt) PVA solution (at 90° C.) were slowly poured into the mixture while stirring at room temperature (emulsion A).

In a separate 600 ml beaker, 100 ml of light paraffin oil and 2 grams of Tween 80 (polyoxyethylene-20-sorbitan monooleate) were mixed at room temperature and stirred at 500 rpm. Eighteen grams of the PEG (at 90° C.) were added to the mixture while stirring at 500 rpm at room temperature (emulsion B).

Subsequently, emulsion B was poured into the beaker containing emulsion A, while stirring. The final mixture was stirred at 500 rpm at room temperature for 1 hour.

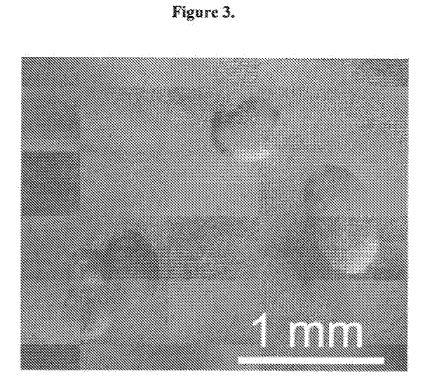

Gel particles were collected from the emulsion mixture and briefly washed with hexane and then repeatedly washed with copious amounts of water. Washed gel particles were well dispersed in water and saline with no further aggregation. Individual particle size was about 0.5 mm and the shape varied from spheres to oblong as shown in FIG. 3.

### Example 5

### Room Temperature Gel Particle Formation in PEG by Atomizer

Ten grams of polyvinyl alcohol (PVA, MW=118,000) are added to 190 g of deionized water and stirred while heating for about 2 hours to prepare a fully dissolved 5% (wt) PVA solution. The dissolved PVA solution is kept in an air convection oven (DKN600, Yamato) at 90° C. for about 16 hours. Polyethylene glycol (PEG, MW=400) was heated to 90° C. in an air convection oven. A rechargeable atomizer sprayer (Type 304, Stainless Steel) is heated to 90° C. by keeping in the air convection oven.

The atomizer is filled with the PVA solution (at. 90° C.) from above, and pressurized to about 1.45 psi with compressed air. Then, PVA solution in the atomizer is sprayed into a container with 100 grams of polyethylene glycol (PEG, MW=400) at room temperature or 7° C. Alternatively, the atomization of PVA solution (at 90° C.) Is carried out into a PEG bath while stirring the PEG at 10 to 10000 rpm.

### Example 6

### Emulsion in Paraffin Oil at 7° C., PVA Injected

Twenty grams of polyvinyl alcohol (PVA, MW=118,000) were added to 180 grams of deionized water and stirred while heating for about 2 hours to prepare a fully dissolved 10% (wt) PVA solution. The dissolved PVA solution was kept in an air convection oven (DKN600, Yamato) at 90° C. for about 16 hours. Polyethylene glycol (PEG, MW=400) was heated to 90° C. in an air convection oven.

Disposable plastic syringes (10 ml) and hypodermic needles (19 gauge, stainless steel) were heated to 90° C. in an air convection oven.

A 500 ml three-neck round bottom flask was immersed in the chiller (Neslab RTE17) and kept at 7° C. throughout the procedure. One hundred milliliters of light paraffin oil were placed in the flask. Then, 2 grams (2% w/v) of Tween 80 (polyoxyethylene-20-sorbitan monooleate) were added to the flask containing the paraffin oil and the mixture was stirred at 350 rpm. The stirring speed was maintained at 350 rpm throughout the procedure. The temperature of the oil mixture was kept at 7° C.

Thirty grams of the previously prepared 10% (wt) PVA solution (at 90° C.) were poured into the plastic syringes and injected through the needle into the flask containing the mixture, while stirring to form an emulsion. Subsequently, 18 grams of the PEG (at 90° C.) were added to the emulsion while stirring. The mixture was then stirred for 30 minutes to ensure the gelation of the PVA domains.

Gel particles were collected from the emulsion mixture and briefly washed with hexane and then repeatedly washed with copious amounts of water.

Washed gel particles were stored in various ways, such as drying in an air convection oven at 40° C., storing in PEG, air, water, or saline at room temperature.

### Example 7

### Freeze-Thawed Emulsion in Paraffin Oil

Twenty grams of polyvinyl alcohol (PVA, MW=118,000) are added to 180 grams of deionized water and stirred while heating for about 2 hours to prepare a fully dissolved 10% (wt) PVA solution. The dissolved PVA solution is kept in an air convection oven (DKN600, Yamato) at 90° C. for about 16 hours. Polyethylene glycol (PEG, MW=400) is heated to 90° C. In an air convection oven.

Disposable plastic syringes (10 ml) and hypodermic needles (19 gauge, stainless steel) are heated to 90° C. in an air convection oven.

One hundred milliliters of light paraffin oil are placed in a 500 ml three-neck round bottom flask at room temperature. Then, 2 grams (2% w/v) of Tween 80 (polyoxyethylene-20-sorbitan monooleate) are added to the flask and the mixture is stirred at 350 rpm.

Thirty grams of the previously prepared 10% (wt) PVA solution (at 90° C.) are poured into the plastic syringes and injected through the needle into the flask containing the mixture, while stirring at room temperature to form an emulsion. Subsequently, 18 grams of the PEG (at 90° C.) are added to the emulsion while stirring.

The flask containing the emulsion is then immediately immersed info a freezer at −20° C. for 1 hour and thawed at room temperature for 1 hour while stirring. Freeze-thaw cycles are repeated as many times as desired. Stirring speed is maintained at 350 rpm.

Gel particles are collected from the emulsion mixture and briefly washed with hexane and repeatedly washed with copious amounts of water.

Washed gel particles are stored in various ways, such as drying in an air convection oven at 40° C., storing in PEG, air, water, or saline at room temperature.

### Example 8

### Room Temperature Emulsion in Paraffin Oil With PVA Injected and Use of Pluronic

Twenty grams of polyvinyl alcohol (PVA, MW=118,000) are added to 180 grams of deionized water and stirred while heating for about 2 hours to prepare a fully dissolved 10% (wt) PVA solution. The dissolved PVA solution is kept in an air convection oven (DKN600, Yamato) at 90° C. for about 16 hours. Polyethylene glycol (PEG, MW=400) is heated to 90° C. in an air convection oven.

Disposable plastic syringes (10 ml) and hypodermic needles (19 gauge, stainless steel) are heated to 90° C. in an air convection oven.

One hundred milliliters of light paraffin oil are placed in a 500 ml three-neck round bottom flask at room temperature. Then, 5 grams of Pluronic L92 (or L81) (BASF) are added to the flask and the mixture is stirred at 350 rpm. The stirring speed is maintained at 350 rpm throughout the procedure.

Thirty grams of the previously prepared 10% (wt) PVA solution (at 90° C.) are poured into the plastic syringes and injected through the needle into the flask containing the mixture, while stirring at room temperature to form an emulsion. Subsequently, 18 grams of the PEG (at 90° C.) are added to the emulsion while stirring. The mixture is then stirred at room temperature for 30 minutes to ensure the gelation of the PVA domains.

Gel particles are collected from the emulsion mixture and briefly washed with hexane and then repeatedly washed with copious amounts of water.

Washed gel particles are stored in various ways, such as drying in an air convection oven at 40° C., storing in PEG, air, water, or saline at room temperature.

### Example 9

### Room Temperature Emulsion in Paraffin Oil With PVA/Nano-Clay Injection

Twenty grams of polyvinyl alcohol (PVA, MW=118,000) are added to 80 grams of 10⁻⁵ M NaOH and stirred while heating for about 2 hours to prepare a fully dissolved 20% (wt) PVA solution. The dissolved PVA solution is kept in an air convection oven (DKN600) at 90° C. for 16 hours.

Two grams of nano-clay, Laponite (Laponite® RD) are dissolved in 98 grams of 10⁻⁵ M NaOH (pH 9) and stirred while heating for 2 hours. The PVA solution is poured into Laponite solution on heat while stirring to generate a 10% (wt) PVA to 1% (wt) Laponite solution.

Polyethylene glycol (PEG, MW=400) is heated to 90° C. in an air convection oven.

Disposable plastic syringes (10 ml) and hypodermic needles (19 gauge, stainless steel) are heated to 90° C. in an air convection oven.

One hundred milliliters of light paraffin oil are placed in a 500 ml three-neck round bottom flask at room temperature. Then, 2 grams (2% w/v) of Tween 80 (polyoxyethylene-20-sorbitan monooleate) are added to the flask and the mixture was stirred at 350 rpm. The stirring speed is maintained at 350 rpm throughout the procedure.

Thirty grams of the previously prepared PVA-Laponite solution (at 90° C.) are poured into the plastic syringes and injected through the needle into the flask containing the mixture, while stirring at room temperature to form an emulsion. Subsequently, 18 grams of the PEG (at 90° C.) are added to the emulsion while stirring. The mixture is then stirred at room temperature for 30 minutes to ensure the gelation of the PVA domains.

Gel particles are collected from the emulsion mixture and briefly washed with hexane and then repeatedly washed with copious amounts of water.

Washed gel particles are stored in various ways, such as drying in an air convection oven at 40° C., storing in PEG, air, water, or saline at room temperature.

### Example 10

### Gel Particle Formation in Gas by Atomizer

Ten grams of polyvinyl alcohol (PVA, MW=118,000) are added to 190 grams of deionized water and stirred while heating for about 2 hours to prepare fully dissolved 5% (wt) PVA solution. The dissolved PVA solution is kept in an air convection oven (DKN600) at 90° C. for 16 hours. A rechargeable atomizer sprayer (Type 304, Stainless Steel) is heated to 90° C. by keeping in the air convection oven.

Thirty grams of the previously prepared 10% (wt) PVA solution (at 90° C.) are placed in a beaker while heating, and 18 grams of the PEG (at 90° C.) are added while stirring. The mixture is stirred on heat for at least 1 minute to insure complete mixing.

The atomizer is filled with the PVA-PEG mixture from above, and pressurized to 145 psi with compressed air. Then, the solution in the atomizer is sprayed into a container with air or helium at 7° C.

### Example 11

### Epichlorohydrin Crosslinking

Thirty grams of polyvinyl alcohol (PVA, MW=118,000) are added to 170 grams of deionized water and stirred while heating for about 2 hours to prepare folly dissolved 15% (wt) PVA solution. Forty grams of 15% PVA solution are placed in a 500 ml three-neck round bottom flask. Subsequently, 4 grams of sodium hydroxide are added to this flask and the solution is stirred at 400 rpm at 50° C. for 1 hour. Six milliliters of Epichlorohydrin (EP) are added to the mixture and stirred at 100 rpm at 50° C. for 10 minutes for pre-crosslinking. Two hundred fifty milliliters of light paraffin oil are placed into foe mixture and vigorously stirred for at least 2 minutes, followed by addition of 5 g of Span 80 (sorbitan monooleate). The mixture is kept stirring at 350 rpm at 50° C. for 24 hours to ensure complete crosslinking of gel particles.

Gel particles are collected from the emulsion mixture and briefly washed with hexane or a mixture of toluene and petroleum ether once and then repeatedly washed with copious amounts of water.

Washed gel particles are stored in various ways, such as drying in an air convection oven (DKN600) at 40° C., storing in PEG, air, water, or saline at room temperature.

### Example 12

### Radiation Crosslinking

Twenty grams of polyvinyl alcohol (PVA, MW=118,000) are added to 180 grams of deionized water and stirred while heating for about 2 hours to prepare folly dissolved 10% (wt) PVA solution. The dissolved PVA solution is kept in an air convection oven (DKN600) at 90° C. for 16 hours.

Disposable plastic syringes (10 ml) and hypodermic needles (19 gauge) are heated to 90° C. in an air convection oven.

One hundred milliliters of light paraffin oil are placed in a 500 ml three-neck round bottom flask at room temperature. Then, 2 grams (2% w/v) of Tween 80 (polyoxyethylene-20-sorbitan monooleate) are added to the flask and the mixture was stirred at 350 rpm.

Thirty grams of the previously prepared 10% (wt) PVA solution (at 90° C.) are poured into the plastic syringes and injected through the needle into the oil flask containing the mixture, while stirring to form an emulsion for 10 minutes. The emulsion mixture is then irradiated using electron beam or gamma radiation while stirring at 350 rpm to crosslink the dispersed gel solution in oil. The radiation dose is at least about 1 kGy, for example, about 25kGy, between 25 and 1000 kGy, about 50 kGy, about 100 kGy, or about 150 kGy.

Radiation-crosslinked gel particles are collected from the emulsion mixture and briefly washed with hexane once and then repeatedly washed with copious amounts of water.

Washed gel particles are stored in various ways, such as drying in an air convection oven at 40° C., storing in PEG, air, water, or saline at room temperature.

### Example 13

### Glutaraldehyde Crosslinking

Twenty grams of polyvinyl alcohol (PVA, MW=118,000) are added to 180 grams of deionized water and stirred while heating for about 2 hours to prepare fully dissolved 10% (wt) PVA solution. The dissolved PVA solution is kept in an air convection oven (DKN600) at 90° C. for 16 hours.

One hundred milliliters of light paraffin oil are placed in a 500 ml three-neck round bottom flask at room temperature. Then, 2 grams (2% w/v) of Tween 80 (polyoxyethylene-20-sorbitan monooleate), 1 ml of 0.1M HCl and 7.5 ml of glutaraldehyde (25% aqueous solution) are added to the flask and the mixture is stirred at 400 rpm. The stirring speed is maintained at 400 rpm throughout the procedure.

Fifty grams of the previously prepared 10% (wt) PVA solution (at 90° C.) are slowly poured into the flask above to form an emulsion, while stirring at room temperature for 30 minutes for crosslinking reaction.

Gel particles are collected from the emulsion mixture and briefly washed with Hexane once and then repeatedly washed with copious amounts of water.

Washed gel particles are stored in various ways, such as drying in an air convection oven at 40° C., storing in PEG, air, water, or saline at room temperature.

### Example 14

### Gelation of PVA Matrix From PEG-Loaded PVA Microparticles (Dual-Barrel Injection)

Twenty grams of polyvinyl alcohol (PVA, MW=118,000) are added to 180 grams of deionized water and stirred while heating for about 2 hours to prepare fully dissolved 10% (wt) PVA solution. The dissolved PVA solution is kept in an air convection oven (DKN60) at 90° C. for 16 hours.

PVA gel particles are prepared following procedures described in Examples 1-10. After immersion in polyethylene glycol (PEG, MW=400) at room temperature for 1 day, particles are drained and centrifuged to remove excess PEG from the particle surface.

The previously prepared 10% PVA solution is placed in one barrel of the disposable plastic dual barrel dispenser, sealed and slowly cooled down to room temperature. Gel particles are placed into the other barrel of dispenser at room temperature. Both the PVA solution and gel particles are injected together through mixer nozzle and hypodermic needles (15 gauge) at room temperature. The injected mixture is kept either at room temperature or 37° C. for gelation. The gelation of the PVA solution that forms the matrix surrounding the particles takes place as the gellant PEG molecules diffuse out of the PEG-loaded PVA gel particles.

### Example 15

### Gelation of PVA Matrix From PEG Loaded PVA Microparticles (Manual Stirring)

Thirty grams of polyvinyl alcohol (PVA, MW=118,000) were added to 170 grams of deionized water and stirred while heating for about 2 hours to prepare fully dissolved 15% (wt) PVA solution. The dissolved PVA solution was kept in an air convection oven (DKN600) at 90° C. for 16 hours.

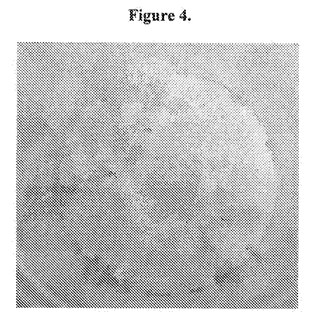

PVA gel particles were prepared following procedures described in Example 4. Gel particles that were dispersed in water were dried at 40° C. for 1 hour to remove excess water on the particle surface. Particles were immersed in polyethylene glycol (PEG, MW=400) while continuously stirring with a magnetic stir bar at room temperature for 1 day. Then particles were drained and 3.5 grams of the particles were placed into a fresh beaker (see FIG. 4).

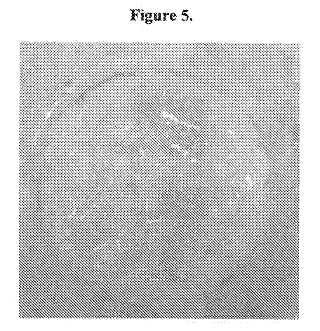

A 5.2 g of the previously prepared 15% PVA solution was poured into over the particles and slightly stirred by a glass stir rod (see FIG. 5).

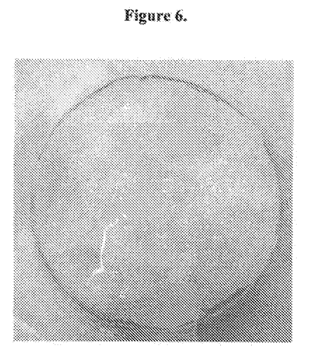

The mixture was covered and kept at mom temperature for 1 day for gelation (see FIG. 6).

### Example 16

### Formation of PVA Fibers Containing Gellant

A 10% (wt) PVA solution (MW: 120,000 g/mole) was prepared by mixing PVA powder in water at 90° C. for 2 hours. The solution was allowed to cool down to room temperature, and was then placed in a syringe. A needle attached to the syringe was suspended vertically in a bath of polyethylene glycol (MW: 400 g/mole) at room temperature. A syringe pump slowly forced the PVA solution through the needle into the bath. While the resulting extruded solution gelled in the PEG bath, it was a wound on a rotating spindle, drawing the extruded filament and entrapping the PEG in the PVA filament.

The filament is then placed in a solution of 10% (wt) PVA in water, where the entrapped PEG gells the PVA precursor solution, and the fiber reinforces the resulting matrix.

### Example 17

### Fabricated PVA Hydrogel Article Containing Polyacrylamide (PAAM) Hydrogel Particles

The PAAM hydrogel particles are fabricated by forming an emulsion of a solution of 12% (wt) acrylamide monomer in water, also containing 1% (wt) N,N′-methylenebisacrylamide, and 1% (wt) oxo-gluteric acid. This mixture is then placed in oil and vigorously stirred to form an emulsion at a concentration of 10% mixture in oil. The emulsion is then exposed to UV light for crosslinking. Once crosslinking is completed the PAAM particles are collected from the emulsion and washed to remove the oil. The PAAM particles are then added to a 15% (wt) aqueous PVA solution. The concentration of the PAAM particles in the PVA solution is about 30% (vol). The concentration of the particles also can be about 1% (vol) or higher, for example, 5% (vol) or higher, 10% (vol) or higher, about 20% (vol) or higher, about 50% (vol) or higher, or about 75% (vol). The PVA solution containing the PAAM particles is then gelled using a gellant, irradiation, or freeze thaw. For example, the PVA solution containing the PAAM particles is heated to 90° C. and mixed with PEG also heated to 90° C. The PEG concentration in the final mixture is 28% (wt). The mixture is then cooled down to room temperature to cause gelation of the PVA matrix around the PAAM particles. The gelation can be done in a desired shaped mold to obtain a finished article with the desired shape.

It is to be understood that, the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

1. A fabricated hydrogel particle comprising at least one type of gellant, wherein the gellant is embedded within the hydrogel particle, and wherein the gellant diffuses out of the hydrogel particle, thereby gelling the surrounding hydrogel matrix.

2-3. (canceled)

4. A fabricated hydrogel composition comprising:
a) fabricated hydrogel particles;
b) at least one type of gellant, wherein the gellant is embedded within the hydrogel particles; and
c) a precursor hydrogel solution, wherein the hydrogel particles are suspended in the hydrogel solution, and wherein the gellant diffuses out of the hydrogel particles, thereby gelling the surrounding hydrogel matrix.

5. A fabricated hydrogel composition comprising:
a) fabricated hydrogel particles, wherein the hydrogel particles are injectable in size;
b) at least one type of gellant, wherein the gellant is embedded within the hydrogel particles; and
c) a hydrogel solution, wherein the hydrogel particles are suspended in the precursor hydrogel solution, and wherein the gellant diffuses out of the hydrogel particles when the composition is injected to a body cavity, thereby gelling the surrounding hydrogel matrix.

6-9. (canceled)

10. A method of making a fabricated hydrogel composition comprising:
a) providing pre-gelled hydrogel particles;
b) loading the hydrogen particles with at least one type of gellant; and
c) providing a precursor hydrogel solution to suspend the hydrogel particles prior to application of the composition formed thereby.

11-12. (canceled)

13. A method of implanting fabricated hydrogel particles into a selected site of a mammal to form a solid implant, wherein the method comprises:
a) implanting a hydrogel composition into a selected site in a mammal, wherein the hydrogel composition comprises fabricated hydrogel particles, at least one type of gellant, wherein the gellant is embedded within the hydrogel particles, and a hydrogel solution in which the hydrogel particles are suspended in, thereby forming a hydrogel matrix; and
b) allowing the gellant to diffuse out of the hydrogel particles, thereby gelling the surrounding of the hydrogel matrix and forming a solid implant.

14. (canceled)

15. The method according to claim 13, wherein the hydrogel particles of a composition comprising the particles are implanted into a mammal m a surgical procedure for intervertebral disc replacement, wound care, cartilage replacement, joint replacement, implantation as a surgical barrier or a gastrointestinal device, a cosmetic and reconstructive operation, or breast or muscle enlargement.

16. The method according to claim 13, wherein the hydrogel particles or a composition comprising the particles are implanted into a mammal to fill-in a cavity in a cartilage defect, in a joint such as hip, knee, or a nuclear cavity, the nuclear space within the intervertebral disc, and act as an articular or load-bearing surface.

17. A method of treating a mammal comprising implanting fabricated hydrogel particles into a selected site of the mammal to form a solid implant, wherein the method comprises:
a) implanting the fabricated hydrogel particles into the selected site of the mammal, wherein the fabricated hydrogel particles comprise at least one type of gellant, wherein the gellant is embedded within the hydrogel particles, and wherein the fabricated hydrogel particles are suspended in a precursor hydrogel solution, thereby forming a hydrogel matrix; and
b) allowing the gellant to diffuse out of the hydrogel particles, thereby gelling the surrounding of the hydrogel matrix and forming a solid implant.

18. (canceled)

19. The method according to claim 17, wherein the mammal is treated for intervertebral disc replacement, wound care, cartilage replacement, joint replacement, implantation as a surgical barrier or a gastrointestinal device, a cosmetic and reconstructive operation, or breast or muscle enlargement.

20. The method according to claim 17, wherein the mammal is treated for a cartilage defect, in a joint such as hip, knee, or a nuclear cavity, the nuclear space within the intervertebral disc, and act as an articular or load-bearing surface.

21. A kit for providing a hydrogel composition to a region of interest comprising: a container of pre-gelled or pre-solidified a hydrogel particles, wherein the hydrogel particles are loaded or embedded with at least one gellant; a container containing hydrogel solution; and a delivery device.

22. (canceled)

23. A method of making a crosslinked hydrogel composition comprising:
a) forming an emulsion of a hydrogel solution in oil, thereby forming pre-solidified or pre-gelled hydrogel particles;
b) cross-linking the hydrogel particles, thereby forming crosslinked hydrogel particles;
c) isolating the crosslinked hydrogel particles from the oil emulsion;
d) loading the crosslinked hydrogen particles with at least one type of gellant; and
e) providing a precursor hydrogel solution to suspend the crosslinked hydrogel particles prior to application of the composition formed thereby.

24-25. (canceled)

26. The method according to claim 23, wherein the hydrogel particles are crosslinked by electron-beam radiation, gamma-radiation, beta-emitters, glutaraldehyde crosslinking, epichlorohydrin (EP) crosslinking, or by photo-initiated crosslinking.

27. The fabricated hydrogel composition of claim 4, wherein the hydrogel comprises domains of different hydrogel particles.

28. (canceled)

29. The fabricated composition of claim 4, wherein PVA hydrogel comprises domains of mechanically deformed hydrogel particles.

30. The fabricated composition of claim 4, wherein the hydrogel comprises oriented domains of hydrogel particles.

31. The fabricated hydrogel particle of claim 1, wherein the hydrogel comprises a polymer, polymer blends, or copolymers selected from the group consisting of polyvinyl alcohol (PVA), polyvinyl pyrrolidone (PVP), alginates, polysaccharides, poly-N-isopropyl acrylamide (PNIAAm), or combinations of two or more thereof.

32. The fabricated hydrogel particle of claim 1, wherein the hydrogel comprises polyvinyl alcohol (PVA).

33. The fabricated hydrogel particle of claim 1, wherein the hydrogel comprises polyvinyl alcohol (PVA) copolymerized and/or blended with at least one of the other polymers.

34. The fabricated hydrogel particle of claim 1, wherein the gellant is selected from the group consisting of salts, alcohols, polyols, amino acids, sugars, proteins, polysaccharides, an aqueous solution thereof, or mixtures of two or more thereof.

35. The fabricated hydrogel particle of claim 1, wherein the gellant is selected from the group consisting of polyethylene glycol (PEG), poly ethylene oxide (PEO), polyvinyl pyrrolidone (PVP), poly-N-isopropyl acrylamide (PNIPAAm), chondroitin sulfate, dextran sulfate, dermatin sulfate and the like, or combinations of two or more thereof.

36. The fabricated hydrogel particle of claim 1, wherein the gellant comprises polyethylene glycol (PEG).

37. The fabricated hydrogel composition of claim 4, wherein the hydrogel solution comprises polyvinyl alcohol (PVA), polyvinyl pyrrolidone (PVP), poly-N-isopropyl acrylamide (PNIAAm), or combinations of two or more thereof.

38. The fabricated fabricated hydrogel composition of claim 4, wherein the hydrogel solution is a polyvinyl alcohol (PVA) solution.

39. The fabricated hydrogel composition of claim 4, wherein the hydrogel particles are spherical, elliptical, or irregular in shape, or mixtures of two or more thereof.

40. The fabricated hydrogel composition of claim 4, wherein the hydrogel particles are agglomerations of a number of particles of a similar or different shapes.

41. The fabricated hydrogel composition of claim 4, wherein the hydrogel particles are used in medical devices and are fabricated by molding the hydrogel particles along with a matrix by mixing gellants with a hydrogel solution outside of a body and prior to implanting the device in the body.

42. The fabricated hydrogel composition of claim 4, wherein the hydrogel particles are used in medical devices and are fabricated and/or molded outside the body, packaged, sterilized, and shipped for use in humans.

43. The fabricated hydrogel composition of claim 41, wherein the medical device comprises domains of the same hydrogel.

44. The fabricated hydrogel composition of claim 41, wherein the medical device comprises domains of different hydrogels.
